SEQUENCE LISTING (2 sequences)

SEQ ID NO: 1 - DNA, 1678 bp, Schizochytrium SC1
actcgtgaggccctttcgcccggcgagggtatgatctggcgggaggaatttggaaaggcagtagcacgtccgttagaaccagaagtgtacgcacgcaaacgcgagcagctcggacataagaagttctcctgggatgagataaatcaacataccaagcgtgacgatctatggatcgttgtcgagggcaaggtgtttgatgtgacccctttcgtagaacgccaccctggtggctggcgtccaattacgcacagtagtggtaaagacggaacagatgcatttagtgaatttcaccccgctagcgtcttggaacgttggatgcctcagtactacatcggtgacgtggacaagtatgaggtttctgccttggtccgcgactttagagccatcaaacaagaactcttggctcgtgggtattttgaaaacaccacctcctattactatgcaaagtacatctggtgcgcttccatgttcgcgccagctctgtatggagtgttgtgctgcacgtcaacatttgcgcatatgctatccgctattggaatggctatgttttggcaacaaatagcttttattggtcatgatgctggccacaacgctgtatctcatgttcgcgatatggatctcttttgggcaggttttatcggtgatatgcttggtggagtggggcttagctggtggaagctgtcccacaacactcaccactgtgtgacaaacagtgtcgagaatgacccagacatccaacacttgccttttctggccattacaaataagctcttcaaacgcttctacagtacattccatgatcgatactttgaggcagatatctttgctcgcttctttgtaggttaccaacacattctgtactatccggtgatgatggttgcacgcttcaatctgattcttcaaagctggctcacccttctttctcgtgaacgtattgactaccgttactcggagatgcttgctcttgctattttctgggtgtggttctataagtttgtcatgtgcttgccgtacaatgagcgtattccatatgttgtgctctcttacgcagttgctggcatcctccatgtccagatctgtatttctcactttatgatggaaactttccacggtcgctctaccgaggaatggattcgtcatcagctgcggacatgtcaggatgtaacatgtccgttttacatggattggtttcatggcggtttgcaatttcagactgagcatcacatgtggccccgcttgccccgtaggaatcttcgggtggcacgtgctcgtctgattgagctctgtgcaaaatacaacctcaattatgttgaaatggactttattgaatcaaacaagcaccttatcagatgcctgcgtaagactgccatggaagcacgtaaactcaagtctggagatgctggattttatgaaagtccaatgtgggaaagtctcaacctccgtggttgagacccctttgaaagctcttgcataatgaaccctgagatgcgcgttcttgcttgcgcattatgacacttcgtccaacctctctcctaagcatgatagggatcgtccgagcttttcaaaatttgaaagtaatgccgatgtcacgtatatgtgtattgggagtagcactggcgttttagagtctagttttttaacaaagccgaaggatgagcgaccgaggcctcgcaatggt

SEQ ID NO: 2 - protein, 472 aa, Schizochytrium SC1
MIWREEFGKAVARPLEPEVYARKREQLGHKKFSWDEINQHTKRDDLWIVVEGKVFDVTPFVERHPGGWRPITHSSGKDGTDAFSEFHPASVLERWMPQYYIGDVDKYEVSALVRDFRAIKQELLARGYFENTTSYYYAKYIWCASMFAPALYGVLCCTSTFAHMLSAIGMAMFWQQIAFIGHDAGHNAVSHVRDMDLFWAGFIGDMLGGVGLSWWKLSHNTHHCVTNSVENDPDIQHLPFLAITNKLFKRFYSTFHDRYFEADIFARFFVGYQHILYYPVMMVARFNLILQSWLTLLSRERIDYRYSEMLALAIFWVWFYKFVMCLPYNERIPYVVLSYAVAGILHVQICISHFMMETFHGRSTEEWIRHQLRTCQDVTCPFYMDWFHGGLQFQTEHHMWPRLPRRNLRVARARLIELCAKYNLNYVEMDFIESNKHLIRCLRKTAMEARKLKSGDAGFYESPMWESLNLRG

(19) United States
(12) Patent Application Publication
(10) Pub. No.: US 2009/0118371 A1
(43) Pub. Date: May 7, 2009

(54) Delta 6 Desaturase From Thraustochytrid and its Uses Thereof

(76) Inventor: Villoo Morawala Patell, Karnataka (IN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/995,985

(22) PCT Filed: Jul. 20, 2006
(86) PCT No.: PCT/IB2006/001988

§ 371 Date: Apr. 21, 2008

(30) Foreign Application Priority Data
Jul. 20, 2005 (IN) 964/CHE/2005

(51) Int. Cl. A61K 31/20; C07H 21/00; C12N 15/64; A61P 43/00; A23K 3/00; C12N 1/15; C12P 7/64

(52) U.S. Cl. 514/558; 536/23.2; 435/320.1; 435/254.2; 435/134; 426/61

(57) ABSTRACT

The present invention is directed to an isolated delta-6 desaturase gene from Schizochytrium. It is further directed to the cloning of delta-6 desaturase derived from Schizochytrium in Yeast. The nucleic acid sequence and the amino acid sequences of the delta-6 desaturase are disclosed. Further disclosed are the constructs, vector comprising the gene encoding the enzyme delta-6 desaturase in functional combination with the heterologous regulatory sequences. The novel delta-6 desaturase can be used in a metabolic pathway to convert linoleic acid to gamma linolenic acid (omega-6 pathway). The invention provides the identification, isolation of these novel nucleic acids from Schizochytrium that encode the above-mentioned proteins. The invention specifically exemplifies recombinant yeast cells harboring the vector comprising the delta-6 desaturase gene and by the virtue of the enzyme produced shall be able to produce gamnia-linolenic acid.

### FIELD OF THE INVENTION

The present invention is directed to a gene delta-6 desaturase isolated from Schizochytrium. It is further directed to the cloning of delta-6 desaturase derived from Schizochytrium in Yeast. The nucleic acid sequence and the amino acid sequences of the delta-6 desaturase are disclosed. Further disclosed are the constructs, vector comprising the gene encoding the enzyme delta-6 desaturase in functional combination with the heterologous regulatory sequences. The novel delta-6 desaturase can be used in a metabolic pathway to convert linoleic acid to gamma linolenic acid (omega-6 pathway). The invention provides the identification, isolation of these novel nucleic acids from Schizochytrium that encode the above-mentioned proteins. The invention specifically exemplifies recombinant yeast cells harboring the vector comprising the delta-6 desaturase gene and by the virtue of the enzyme produced shall be able to produce gamma-linolenic acid. The polyunsaturated fatty acids produced by use of the enzyme may be added to pharmaceutical compositions, nutritional compositions, animal feeds, as well as other products such as cosmetics.

### BACKGROUND OF THE INVENTION

Delta-6 desaturases are the key enzymes required for the synthesis of highly unsaturated fatty acids such as Arachidonic acid, docosahexaenoic acid. The major metabolite product of the n-6 pathway is arachidonic acid (20:4n-6), whilst the major end products of the n-3 pathway are eicosapentanoic acid (EPA) (20:5n-3) and docosahexaenoic acid (DHA) (22:6n-3). The availability of 20- and 22-carbon (n-6) and (n-3) polyenoic fatty acids is greatly dependant upon the rate of desaturation of 18:2(n-6) and 18:3 (n-3) by delta-6 desaturase. Delta-6 desaturase is a microsomal enzyme and is thought to be component of a three-enzyme system that includes NADH-cytochrome b5 reductase, cytochrome b5 and delta-6 desaturase. Delta-6 desaturases catalyses the first and the rate limiting step of the PUFA synthesis. It acts as a gateway for the flow of fatty acids through the desaturation and the elongation pathway. Although it can act on any long chain fatty acid, the substrate binding affinity increases greatly with the number of double bonds already present. Recent identification of a human case of delta-6 desaturase deficiency underscores the importance of this pathway (Nakamura et al., 2003).

Unsaturated fatty acids such as linoleic acid and alpha-linoleic acid are essentially dietary constituents that cannot be synthesized by vertebrates since the vertebrate cells can introduce double bonds at the delta-9 position of the fatty acids but cannot introduce additional double bonds between the delta-9 and the methyl terminus of the fatty acid. Hence it is evident that animals cannot desaturate beyond the Delta-9 position and therefore cannot convert oleic acid to linoleic acid, likewise gamma-linolenic acid cannot be synthesized by mammals. Because they are precursors of other products, linoleic and alpha-linoleic acid are essential fatty acids (cannot be synthesized by the body and hence require to form a part of diet), and are usually obtained from plant sources. Linoleic acid can be converted by mammals into gamma-linolenic acid, which can in turn be converted to arachidonic acid (20:4), a critically important fatty acid since it is an essential precursor of most prostaglandins. Furthermore, animal bioconversions of high polyunsaturated fatty acids from linoleic, alpha-linolenic and oleic acids are mainly modulated by the delta6 and delta5 desaturases through dietary and hormonal stimulated mechanisms. (Prostaglandins Leukot Essent Fatty Acids 68(2): 151-62.).

In view of the foregoing, there exists a definite need for the enzyme delta-6 desaturase, the respective genes for encoding this enzyme, including recombinant methods of producing this enzyme. The current requirement for these essential fatty acids have been satisfied through the dietary intake of plant sources rich in such PUFAs. But disadvantages do exist as these natural sources are always subjected to uncontrollable fluctuations in availability. Moreover, plant oils possess a highly heterogenous composition, requiring extensive purifications procedures to separate a particular polyunsaturated fatty acid of interest (US 20060035351). However, cost effective alternatives have to be explored for fulfilling the needs of the growing global populations.

The subject invention relates to the introduction of genes encoding the enzyme delta-6 desaturase isolated from the marine organism Schizochytrium in to yeast for the production of fatty acids such as gamma-linolenic acid, stearidonic acid and the other fatty acids resulting from the bioconversions of the respective substrates in the omega-3/omega-6 fatty acid biosynthetic pathway. Yeast provides numerous advantages as a favorable system for the expression of the fatty acid in a suitable medium. Yeast has long been recognized and used as a host for protein expression since it can offer the processing system along with the ease of use of microbial systems. As a host, it boasts of a number of benefits as it can be used for the production of both secreted and cytosolic proteins which may require post-translational modifications and its biosynthetic pathway resembles higher eukaryotic cells in many aspects. Moreover, in comparison to the other eukaryotic systems, there is considerably more advanced understanding of its genetics with an ease of manipulation similar to that of E. coli. The expression levels also range to several milligrams per liter of the culture.

A number of delta-6 desaturases have been identified. In plants such as the herb, borage (Borago officianalis), the delta-6 desaturase has been identified (Sayanova et al., 1997). The same has been identified in humans (Hyekyung et al., 1999), in animals such as nematode, Caenorhabditis elegans (Michaelson et al., 1998 and Napier et al., 1998) and in Eukaryotic microorganisms such as fungus Mortierella alpina (Hunag et al., 1999 and Knutzon et al., 1998). According to the aspects of the present invention there is provided an isolated nucleic acid molecule comprising the DNA sequence encoding for the enzyme delta-6 desaturase isolated from the marine organism Schizochytrium.

### SUMMARY OF THE INVENTION

The present invention relates to an isolated nucleic acid sequence or fragment thereof encoding a polypeptide molecule possessing desaturase activity, the nucleic acid sequence of which has been represented in SEQ ID. No. 1 and amino acid sequence of which has been represented in SEQ ID. No. 2.

The present invention encompasses an isolated nucleic acid sequence or fragment thereof comprising, or complementary to, a nucleic acid sequence having at least 70%, preferably 80% and more preferably 90% nucleotide sequence identity to a nucleotide sequence represented in SEQ ID. No. 1.

The present invention also includes an isolated nucleic acid sequence or fragment thereof encoding a polypeptide having desaturase activity, wherein said polypeptide comprises an amino acid sequence having at least 70%, preferably 80% and more preferably 90% amino acid sequence identity to an amino acid sequence represented in SEQ ID. No. 2.

The nucleotide sequences described above encode a functionally active Delta-6-desaturase that utilizes a monounsaturated or polyunsaturated fatty acid as a substrate. The nucleotide sequences have be isolated from Schizochytrium SC-1.

Additionally, the present invention includes a method of identification, isolation and cloning of the nucleic acid sequence and amino acid sequence encoding delta-6 desaturase comprising the steps of (1) cDNA library screening with a partial delta-4 desaturase gene leading to the identification of a partial cDNA clone (2) Using the partial cDNA clone for screening the BAC library of Schizochytrium SC-1 for identification of a positive BAC clone (3) Identification and sequencing of the positive BAC clone and further identification of the delta-6 desaturase ORF within the full length sequence (4) constructing a vector comprising the at least 90% sequence identity to the sequence represented in SEQ ID 1 (5) Introducing the constructed vector via transformation into a host cell for a time and under conditions sufficient for the expression of the desaturase.

The host cell may be for example, a eukaryotic cell or a prokaryotic cell. A prokaryotic cells may be for example E. Coli and a prokaryotic cell may be for example a fungal cell, insect cell, mammalian cell or a plant cell but preferably a yeast cell such as Saccharomyces cerevisiae. Other suitable host cells may include Yarrowia lipolytica, Candida sp, Hansenula spp etc,

A particular embodiment of the invention describes the construction of the vector comprising the nucleotide sequence or fragment thereof encoding polypeptide having delta-6 desaturase activity, wherein the said polypeptide comprises an amino acid sequence having at least 70%, preferably 80% and more preferably 90% amino acid sequence identity to the sequence of SEQ ID. NO. 2, operably linked to a regulatory sequence (eg., promoter and terminator) under optimal conditions for the expression of the enzyme delta-6 desaturase.

Additionally, the invention includes a yeast cell comprising the above vector, wherein the expression of the enzyme delta-6 desaturase results in the production of gamma-linolenic acid.

Yet another aspect of the invention relates to induction of the yeast clone expressing delta-12 and delta-6 desaturases, showing the formation of linoleic acid and gamma linolenic acid. The in-vivo conversion of oleic acid to linoleic acid is carried out by Brassica juncae delta-12 desaturase. The subsequent desaturation of linoleic acid to gamma linolenic acid is catalyzed by the cloned SC-1 delta-6 desaturase. In the context of the said invention the experiment demonstrates the functional expression of SC-1 delta-6 desaturase in yeast.

### DETAILED DESCRIPTION OF THE FIGURES AND SEQUENCES

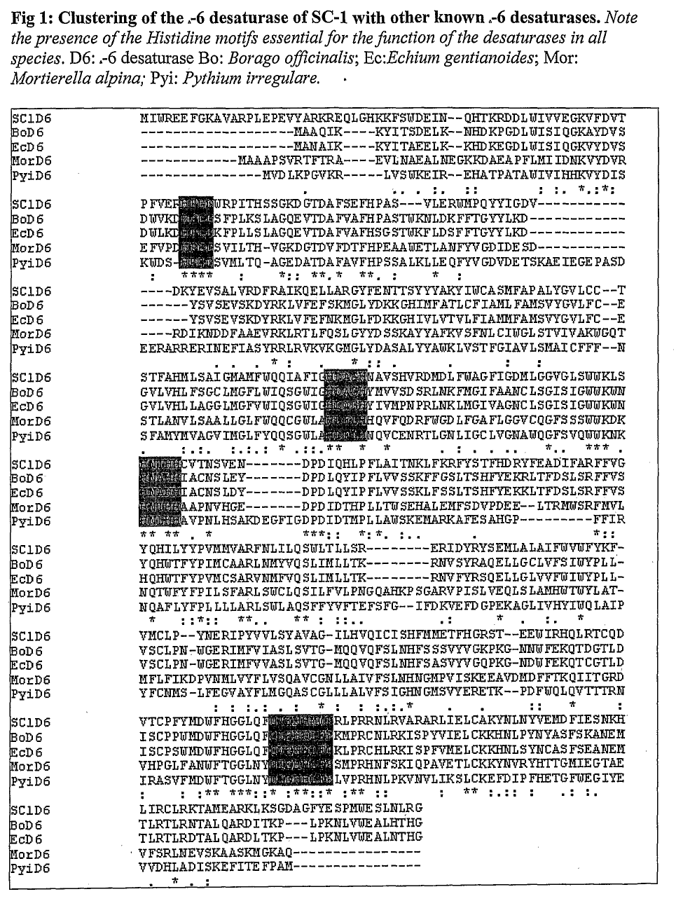

FIG. 1: Clustering of the Delta-6 desaturase of SC-1 with other known Delta-6 desaturases. (Note the presence of the Histidine motifs essential for the function of the desaturases in all species.)

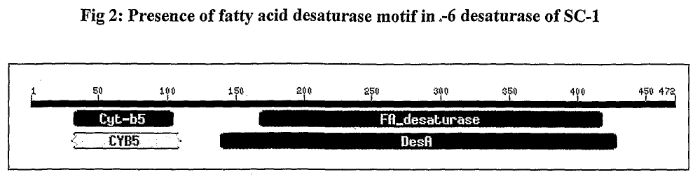

FIG. 2: Presence of fatty acid desaturase motif and Cytocrome B-5 domain in Delta-6 desaturase of SC-1.

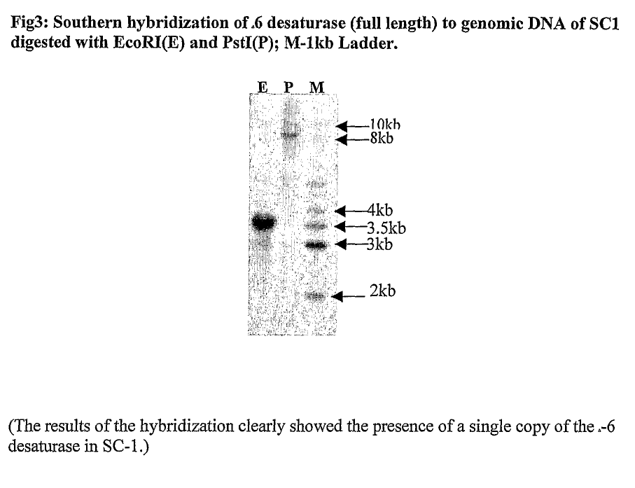

FIG. 3: Southern hybridization of Delta-6 desaturase (full length) to genomic DNA of SC1 digested with EcoRI(E) and PstI(P); M-1 kb Ladder. (The results of the hybridization clearly showed the presence of a single copy of the □-6 desaturase in SC-1.)

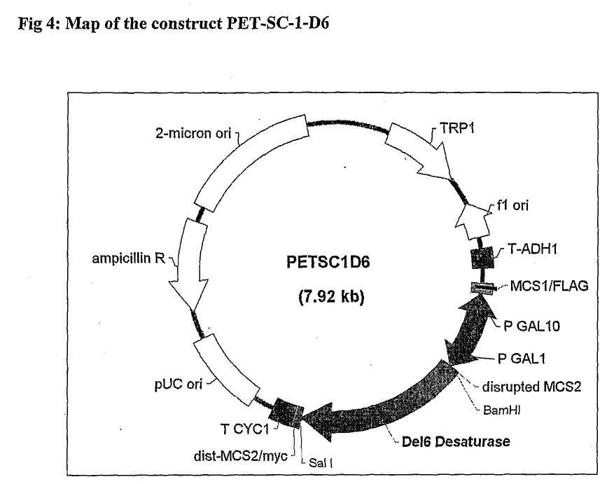

FIG. 4: Map of the construct PET-SC-1-D6.

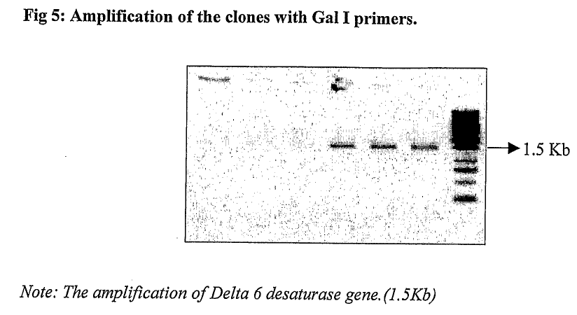

FIG. 5: Amplification of the clones with Gal I primers. (Note: The amplification of Delta 6 desaturase gene. (1.5 Kb))

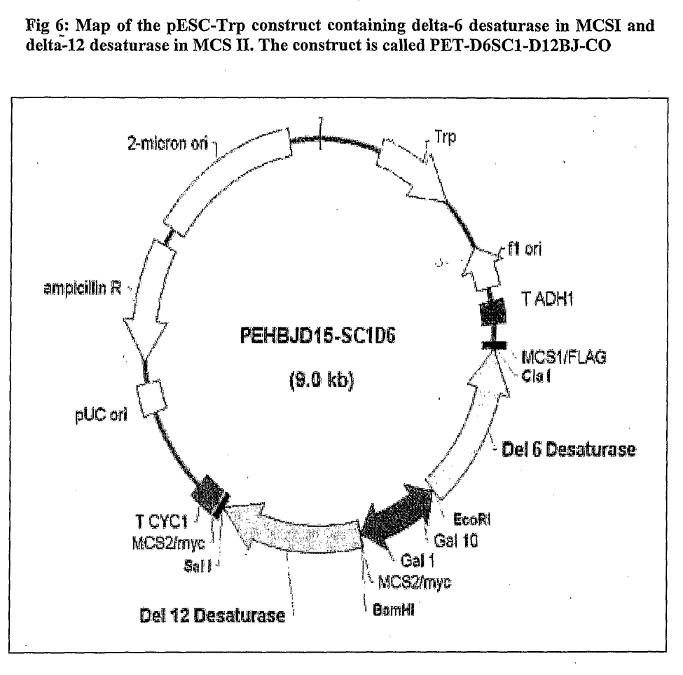

FIG. 6: Map of the pESC-Trp construct containing Delta-6 desaturase in MCSI and Delta-12 desaturase in MCS II. The construct is called PET-D6SC1-D12BJ-CO.

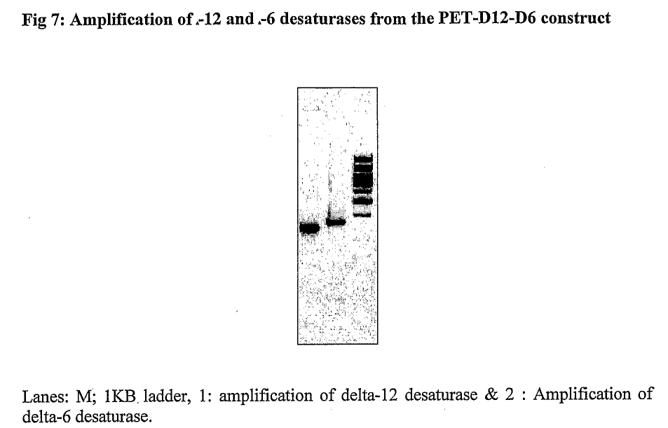

FIG. 7: Amplification of □-12 and □-6 desaturases from the PET-D12-D6 construct (Lanes: M; 1 KB ladder, 1: amplification of □-12 desaturase & 2: Amplification of □-6 desaturase.)

SEQ ID. No. 1: Nucleic Acid Sequence of Delta-6-desaturase isolated from Schizochytrium SC1

SEQ ID. No. 2: Amino Acid Sequence of Delta-6-saturase isolated from Schizochytrium SC1.

### DETAILED DESCRIPTION OF THE INVENTION

Linoleic acid is converted to gamma-linolenic acid by the enzyme delta-6 desaturase. The subject invention relates to an isolated nucleic acid sequence encoding delta-6 desaturase. It more specifically refers to the nucleotide and the corresponding amino acid sequences from the delta-6 desaturase genes derived from the marine organism Schizochytrium obtained through the screening of the BAC library of Schizochytrium.

The invention further relates to the transfer of the vector comprising the nucleic acid fragments of the invention or a part thereof that encodes a functional enzyme along with the suitable regulatory sequences that direct the transcription of their mRNA, into a living cell, which under the context of the present invention is a yeast cell thereby resulting in the production of the specified delta-6 desaturase leading to the conversion of linoleic acid to gamma-linolenic acid.

In the context of this disclosure, a number of terms shall be used. The following definitions are provided to better define the present invention and guide those of ordinary skill in the art in the practice of the present invention. Unless otherwise noted, terms are to be understood according to conventional usage by those of ordinary skill in the relevant art.

Desaturase: Desaturase is an enzyme that promotes the formation of a carbon-carbon double bonds in a hydrocarbon molecule.

Fatty acid desaturase: The term “fatty acid desaturase” used herein refers to an enzyme which catalyzes the breakage of a carbon-hydrogen bond and the introduction of a carbon-carbon double bond into a fatty acid molecule. The fatty acid may be free or esterified to another molecule including, but not limited to, acyl-carrier protein, co-enzyme A, sterols and the glycerol moiety of glycerolipids.

“Delta-6 desaturase” refers to a fatty acid desaturase that catalyzes the formation of a double bond between carbon positions 12 and 13 (numbered from the methyl end), i.e., those that correspond to carbon positions 6 and 7 (numbered from the carbonyl carbon) of an 18 carbon-long fatty acyl chain. As described herein and under the context of the present invention, delta-6 desaturase catalyses the conversion of linoleic acid to gamma-linolenic acid.

“Isolated nucleic acid fragment or sequence” is a polymer of RNA that is single- or double-stranded, may optionally contain synthetic, non-natural or altered nucleotide bases. An isolated nucleic acid fragment in the form of a polymer of DNA may be comprised of one or more segments of cDNA, genomic DNA or synthetic DNA.

Recombinant nucleic acid: A sequence that is not naturally occurring or has a sequence that is made by an artificial sequence that is made by an artificial combination of two otherwise separated segments of sequence. This artificial combination is often accomplished by chemical synthesis or, more commonly, by the artificial manipulation of isolated segments of nucleic acids eg., by the genetic engineering techniques such as those described in Sambrook et al. Molecular Cloning: A Laboratory Manual, 2rd Edition, Cold Spring Harbor Laboratory press, NY, 1989.

“Gene” refers to a nucleic acid fragment that expresses a specific protein, including regulatory sequences preceding (5′ non-coding sequences) and following (3′ non-coding sequences)

“Promoter” refers to a DNA sequence capable of controlling the expression of a coding sequence or functional RNA.

“Coding sequence” refers to a DNA sequence that codes for a specific protein and excludes the non-coding sequences. It may constitute an “uninterrupted coding sequence” i.e., lacking an intron or it may include one or more introns bounded by appropriate splice junctions.

“Initiation Codon” and “Termination Codon” refers to the unit of three adjacent nucleotides in a coding sequence that specifies initiation and chain termination respectively, of protein synthesis (mRNA translation).

“Open Reading Frame” (ORF) refers to the coding sequence uninterrupted by introns between initiation and termination codons that encodes an amino acid sequence.

“Operably linked” refers to the association of nucleic acid fragment so that the function of one is regulated by the other.

“Homologs” Two nucleotide or amino acid sequences that share a common ancestral sequence and diverged when a species carrying that ancestral sequence spilt into two species. Homologs frequently show a substantial degree of sequence identity.

“Transformation” herein refers to the transfer of a foreign gene into the genome of a host organism and its genetically stable inheritance.

“Expression”, as used herein refers to the transcription and stable accumulation of sense (mRNA) or antisense RNA derived from the nucleic acid fragments of the invention. Expression also refers to the translation of mRNA into a polypeptide.

The terms “plasmid”, “vector”, and “cassette” refers to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double stranded DNA or RNA, derived from any source, in which a number of nucleotide sequences have been joined or recombined into a unique construction that is capable of introducing a promoter fragment and DNA sequence for a selected gene product along with appropriate 3′ untranslated sequence into a cell. “Expression cassette” refers to a specific vector containing a foreign gene and having elements in addition to the foreign gene that allow for enhanced expression of that gene in a foreign host.

In accordance with one aspect of the present invention, the cDNA library of Schizochytrium (SC1) (herein after referred as “SC1”) has been screened with a partial delta-4 desaturase gene. This has lead to the identification of a clone of 617 base pair length homologous to the delta-6 desaturase gene of various other organisms. The identified clone is a partial cDNA clone.

In accordance with another aspect of the present invention, the partial clone identified was used to screen the BAC library of SC1. Screening the BAC library lead to the identification of a positive clone comprising the full length sequence of the delta-6 desaturase gene. The clone was further sequenced and the delta-6 desaturase ORF (open reading frame) was identified within the sequence.

The nucleic acid sequence of the delta-6 desaturase has been represented in SEQ ID 1. The nucleic acid sequence translates into a protein of 472 amino acids. The amino acid sequence of the delta-6 desaturase from SC-1 has been represented in SEQ ID 2. The invention encompasses other “obtainable” delta-6 desaturases from other organisms such as SC-1. “Obtainable” refers to those desaturases, which have sufficiently similar sequences to that of the sequences provided herein that encodes a biologically active protein.

In yet another aspect of the invention, the degree of homology of the isolated delta-6 desaturase is compared with the delta-6 desaturase of different species. The nucleic acid sequence of the isolated delta-6 desaturase is compared to “homologous” or “related” to DNA sequences encoding delta-6 desaturases from other organisms. “Homologous” or “related” includes those nucleic acid sequences, which are identical or conservatively substituted as compared to the exemplified organisms such as Borago officinalis, Echium gentianoides, Mortierella alpina, and Pythium irregulare. The similarity between two nucleic acids or two amino acid sequences is expressed in terms of percentage sequence identity. The higher the percentage sequence identity between the two sequences, the more similar the two sequences are. Sequences are aligned, with allowances for gaps in alignment, and regions of identity are quantified using a computerized algorithm. Default parameters of the computer programs are commonly used to set gaps allowances and other variables.

Methods of alignment of sequences are well known in art. Various programs and alignment algorithms are described by Pearson et. al., Methods in Molecular Biology 24:307-331, 1994 and in Altschul et al., Nature Genetics. 6:119-129, 1994. Altschul et al presents a detailed consideration of sequence alignment methods and homology calculations. The NCBI Basic Local Alignment Search Tool (BLAST) (Altschul et al., J. Mol. Biol. 215:403-410, 1990 is available from several sources, including the National Center of Biotechnological Information (NCBI, Bethesda, Md.) and on the internet, or use in connection with the sequence analysis programs blastp, blastn, blastx, tblastn, and tblastx etc.

Additionally, it will be appreciated by one skilled in art that polypeptides may have certain amino acids conservatively substituted in a manner such that the function of the polypeptide is not altered or comprised. It is very evident from the comparative homology conducted as represented in FIG. 1 that the histidine motifs have been conserved over the organisms compared.

In another aspect of the present invention, the delta-6 desaturase sequence was subjected to a motif search for confirmation of the presence of the desaturase domain. The results of motif search is represented in FIG. 2. It was hence confirmed that the gene has the complete desaturase domain and the cytochrome b5 domain characteristic of the functional desaturases.

Recombinant nucleic acids, as mentioned for instance in SEQ ID: 1, containing all or a portion of the disclosed nucleic acid operably linked to another nucleic acid element such as promoter, for instance, as part of a clone designed to express a protein. Cloning and expression systems are commercially available for such purposes. Vectors containing DNA encoding the delta-6 desaturase are also provided by the present invention.

Various host cells can be used for expression of the protein. For example, various yeast strains and yeast-derived vectors are commonly used for expressing and purifying proteins. The current invention uses Saccharomyces cerevisiae as the host for the expression of the cloned gene. But also envisaged is the usage of other expression systems such as the Pichia pastoris expression systems.

Vectors or DNA cassettes useful for the transformation of suitable host cells are well known in art. Typically, however, the vector or cassette contains sequences directing transcription and translation of the relevant gene(s), a selectable marker Expression vectors such as pET systems can be used to express the gene of interest. The vector may be a plasmid, cosmid or bacteriophage preferably for the purposes of the invention a plasmid, may comprise the nucleotide sequence (eg. Promoter) which is functional in the host cell and is able to elicit expression of the desaturase encoded by the nucleotide sequence. (The promoter is “operably linked” with the coding sequence). Some suitable promoters include genes encoding T7, TPI, lactase, metallathionein or promoters activated in the presence of galactose such as GAL1 and GAL10. The kind of promoters used for expression shall depend upon the kind of expression product desired and also the nature of the host cell. For example in the current invention GAL1 or GAL10 promoters are used to control the expression of the delta-6 desaturase gene sequences. Any one of a number of regulatory sequences can be used, depending upon whether constitutive or induced transcription is desired, the efficiency of the promoter expressing the ORF of interest, the ease of construction and the like. Nucleotide sequences surrounding the translational initiation codon ‘ATG’ have been found to affect expression in yeast cells and certain nucleotide sequences of exogenous genes can be modified for desired expression levels. For expression in yeast, this can be done by site-directed mutagenesis of an inefficiently expressed gene by fusing it in-frame to an endogenous yeast gene, preferably a highly expressed gene.

Useful selectable markers can be used for the selection of the successfully transformed cells post transformation. Selectable markers for selection are not limited to streptomycin, Ampicillin etc.

The vector constructed may be then introduced into the host cell of choice by the methods known to those ordinary skilled in art such as transfection, electroporation or transformation. Such techniques of have been well illustrated in Molecular Cloning: A laboratory Manual. Vol 1-3 Sambrook et. al., Cold Spring Harbor Laboratory Press (1989). The host cell that has taken up the expression cassette that has been manipulated by any method to take up a DNA sequence will be herein referred to as “transformed” or “recombinant”.

The present invention is further illustrated in the following examples. It should be understood that these examples, while indicating preferred embodiments of the invention, are given by way of illustration only. From the above discussion and these examples, one skilled in the art can ascertain the essential characteristics of the invention and without departing from the spirit and scope thereof, can make variouis changes and modifications of the invention to adapt it to various usages and conditions.

### EXAMPLES

### Example 1

Screening of the cDNA Library of SC1 With Partial Delta-4-Desaturase Gene:

Screening of the cDNA library of SC-l with the partial A4 desaturase gene obtained from the sequencing of the SC-1 cDNA library led to the identification of a number of clones. One of these clones of 617 bp was found to be homologous to 6 desaturase of several organisms.

The sequence had an ORF running through till 273 bases. The 3′UTR is 401 bases A polyadenylation signal “AATAA” is seen towards the 3′ end of the sequence.

This sequence when subjected to homology search against the protein database of NCBI shows homology to −6 desaturases of Echium plantagina, Aragania spinosa and Echium pitardii v.

The protocols involved were

(A) Protocol for Plating of cDNA Library and Transfer to Membrane

Serial Dilutions

1 μl of cDNA library clone mix and 9 μl of SOC were taken into an eppendorf (dilution factor 10-1), and the tube was labeled as A. From tube A, 1 μl of clone mix and add 9 μl of Soc was taken into another fresh tube, labeled as B (dilution factor 10-2). From tube B 1 μl of clone mix was taken and 9 μl of SOC was added into another fresh tube, labeled as C. 1 micro litre from tube A, B, & C was taken and 99 μl of SOC was added.

Plating

1. 100 μl of final clones mix from each tube was plated to separate LB amp plates.

2. The plates were incubated at 37° C. overnight.

3. The plate that had 104 cells/plate or more was taken for transfer.

Transfer on to the Membrane
1. The plates were marked with Indian ink at four places, for proper orientation of the clones.
2. The nylon membrane was inverted on to the plate and allowed to soak for 1-2 min.
3. The membrane was lifted from one side with a sterile forceps and was then air-dried and further taken up for hybridization.

(B) Protocol for Preparation of Labeled Probes by Random Priming
1. The DNA for labeling was dissolved in either sterile water or 10 mMTris HCl (pH-8.0), 1 mM EDTA to a concentration of 10 μg/ml.
2. The DNA was denatured at 95° C. for 2 minutes (by keeping the vial containing the DNA in boiling water bath) & chilled immediately on ice.
3. Reagents were added in the following order in a small eppendorff vial kept on ice to label 50 ng of DNA:
5 μl of denatured DNA was taken in to the vial; to this 5 μl of random primer buffer was added, then 5 μl of random primer solution was added, further to which 12 μl of dNTP mix, 2 μl of klenow enzyme (1U/μl ), 18 μl of sterile water were added.
4. The tube was capped and mix gently either by slowly tapping at the bottom or by a ‘tap spin’, in a centrifuge.
5. 3 μl (30 μCi) of P32 labeled nucleotide was added to the above mix, by placing the tube behind the acrylic shield.
6. The tube was placed in a constant temperature at 37° C. in a PCR block.
7. The tube was then kept at 95° C. for 15 min in a PCR block and chilled immediately on ice.
8. The Random labeled fragment was ready for probing.

(C) Protocol for Hybridization
1. 25.0 ml of Pre-Hybridisation buffer was taken in the hybridization bottle and the membrane was immersed into it.
2. The bottle was then placed in the hybridization oven set at 65° C. for 2 hrs
3. The pre-hybridisation buffer was discarded and 25.0 ml of fresh pre-hybridisation buffer was added.
4. 50 μl of random labeled probe was added to the bottle behind the acrylic shield.
5. The bottle placed back in the hybridization oven set at 65° C. overnight.
6. The solution-containing probe was decanted into a labeled, radioactive discard can for disposal.
7. The membrane was rinsed with 2×SSC at room temperature to remove any unbound probe.
8. The membrane was further washed with 2×SSC+0.1% SDS at 650 C for 15 min on a rocker in the oven.

### Example 2

Construction and Screening of BAC Library With the Delta-6 Desaturase Partial cDNA Clone of SC-1:

Screening of the BAC library of SC-1 with one of the partial clones led to the identification of a positive BAC clone. The BAC clone was sequenced and the −6 desaturase ORF identified within the sequence.

Protocols for BAC Library Construction:

DNA purified by Pulse field gel electrophoresis was digested with restriction enzyme 1 unit of Eco RI wherein fragments of 75-200 kb were maximally obtained. The size selected DNA was ligated (100 units of high concentration T4 DNA ligase (400 μ/microl; NEB biolabs) with 1:10::Insert:vector molar ratio) to the digested BAC vector (pIndigoBAC536) and transformed by electroporation in E. coli electrocompetant cells and plated on suitable medium. The recombinant clones would be picked and inoculated in SOB in a 96 well plate and the library is stored at −70° C. as glycerol stocks.

The protocols for screening of the BAC library are same as described in Example 1.

The sequence shows a high degree of homology to the −6 desaturase of different species.

The −6 desaturase sequence when subjected to a motif search, showed that the gene has the complete desaturase domain and the Cytochrome b5 domain characteristic of the functional desaturases.

### Example 3

Determination of the Gene Copy No:

10 μg of genomic DNA isolated from SC-1 was digested with Eco RI or Pst I, and was loaded on 0.8% agarose gel, electrophoresed at 30 volts overnight and the DNA was transferred to nylon N+ membrane (milipore). The SC-1 delta-6 desaturase gene labeled with ³²PdCTP by random priming was hybridized to the blot at 65° C. overnight. The blot was then washed with moderate stringency (2×SSC-15 min, 2×SSC+0.1%SDS-15 min, 0.5×SSC+0.1%SDS-15 min at 65° C.) and exposed to X-ray film.

The results of the hybridization have been represented in FIG. 3 and the results of the hybridization clearly showed the presence of a single copy of the delta-6 desaturase in SC-1. Cross hybridizing homologous sequences did not occur in the SC-1 genome.

### Example 4

Construction of the Vector:

The delta-6 desaturase gene was cloned into the MCSII site under the GAL1 promoter between the BamHI and the SalI sites of pESC-Trp (PET-SC 1-D6). Primers used for the amplification are given below.

| | |
|---|---|
| D6 pES F | CGGGATCCTATGATCTGGCGGGAGG |
| D6 pES R | ACGCGTCGACTCAACCACGGAGGTTGAGAC |

Table 1: Primers synthesized for the amplification and cloning of delta-6 desaturase from SC1 into the MCSII of pESC between BamHI and Sal I sites. The restriction sites in the primers are given in red.

PCR components for 20 ul reaction

| | |
|---|---|
| Milli-Q water upto | 20, 1 |
| 10X reaction buffer | 2.0, 1 |
| dNTP mix (1OmM) | 0.2, 1 |
| Forward Primer (5.0 picomoles/ul)/ | 1.0, 1 |
| Reverse Primer (5.0 picomoles/ul)/ | 1.0, 1 |
| Genomic DNA of Sc-1 (100 ng) | 1.0, 1 |
| Taq polymerase (3 U/ul) | 0.1, 1 (~0.3 U) |

The cycling conditions are as follows:

| | | | | |
|---|---|---|---|---|
| 94° C. | 94° C. | 55° C. | 72° C. | 72° C. |
| 3 minutes | 30 seconds | 30 seconds | 1.3 minute | 7 minutes |

| | | |
|---|---|---|
| 1 cycle | 35 cycles | 1 cycle |

The ORF of the delta-6 desaturase has been amplified with the above primers, restricted with Bam HI and Sal I and directionally cloned into the corresponding sites of pESC-Trp. The construct has been named PET-SC-1-D6 and is represented in FIG. 4.

### Example 5

Transformation of Yeast:

The construct as represented in FIG. 4 was been transformed into Saccharomyces cerevicea YPH500 strain and the transformants were confirmed by PCRs. The PCR results are represented in FIG. 5. Amplification of the clones (Kit used is from Stratagene, Yeast Epitope Tagging Vector) with Gal I primers indicated the Delta-6-desaturase gene.

Protocol for Preparation of Yeast Competent Cells:

All the steps are to be carried out in aseptic conditions. A single colony is inoculated into YPD and grown overnight at 30° C. Using 5% of inoculum a 50 ml culture was grown at 30° C. till the O.D reaches 1.0. The cells are left on ice for 10 min and centrifuged at 5000 rpm for 10 min at 4° C. and the media is discarded. The pellet is resuspended in equal volume of water (50 ml) and spun at 5000 rpm for 10 min at 4° C. The pellet was washed twice in equal volume of 1 M sorbitol and centrifuged at 5000 rpm for 10 min at 4° C. Finally the pellet was resuspended in 150 μl of 1 M sorbitol and stored at 4° C. The competent cells can be stored for a week.

Transformation of Yeast by Electroporation:

60 μl of the competent cells and ˜1 μg of DNA were taken in a vial, mixed and kept on ice. This was further taken onto a 0.2 cm electroporation cuvette and given a pulse set at SC2 (1.7 kV and 5.8 ms). Immediately 600 μl of 1 M sorbitol was added and the cells were resuspended and transfered into a vial and stored at room temperature for 5 min. 200 μl of cells were spread on a suitable selection medium and incubated at 30° C. for 2 days. The number of colonies expected were 100 per 200 μl of culture spread.

The transformed yeast cells were selected by growing them in SD Dropout Media with. Tryptophan. (Sigma).

### Example 6

In-Vivo Proof of Function

The in-vivo proof of function experiment was performed in yeast strain YPH 499 transformed with pESC-Trp construct containing Delta-6 desaturase and Brassica juncae delta-12 desaturase. Using this construct the in-vivo Delta-6 desaturase activity can be observed in absence of addition of precursor fatty acid in the media. The −6 desaturase cloned between the Eco RI and Spe I sites of MCS I of the pESC-Trp was restricted with Bam HI and Sal I. The PEH-D12-BJ-CO clone carrying Delta-12 desaturase was digested with BamHI and Sal I and the Delta-12 desaturase thus released was isolated. The latter was directionally cloned into the corresponding sites MCSII of the above construct. The construct thus obtained has delta-6 in MCSI and Delta-12 in MCS II. The above construct is called as PET-D6 SC1-D12BJ-CO (FIG. 6.)

The presence of both the genes in some of the selected clones was confirmed by PCR amplification and sequencing. (FIG. 7.)

The recombinant clones were grown overnight in SD medium without tryptophan (0.67% yeast N2 base W/O amino acids; 2% Dextrose; 0.13% amino acid drop out powder without tryptophan). The cells were pelleted at 5,000 rpm for 10 minutes, washed once with sterile water and resuspended in SG medium without tryptophan 0.67% yeast N2 base W/O amino acids; 2% galactose; 0.13% amino acid drop out powder without tryptophan). The cultures were incubated at 30 C for 1 day; the cells were pelleted, lyophilized. For fatty acid profiling, lipid extraction was performed and fatty acid methyl esters (FAME) were prepared and analyzed using GC-MS. The fatty acid profile of a typical recombinant yeast clone is given in the table below.

TABLE
Fatty acid analysis of yeast expressing
Delta-12 and Delta-6 desaturases

Fatty acid composition (GC %)

| Fatty acids | pESC | Delta-12 + Delta-6 desaturase |
|---|---|---|
| 14:0 | 0.7 | 0.3 |
| 16:0 | 19.6 | 18.3 |
| 16:1 | 38.4 | 33.6 |
| 16:2 | — | 4.4 |
| 18:0 | 5.8 | 6.4 |
| 18:1 | 35.5 | 26.4 |
| 18:2 | — | 9.7 |
| 18:3* | — | 0.8 |

*gamma linolenic acid

It is evident from the table above that upon induction, the yeast clone expressing delta-12 and delta-6 desaturases shows the formation of linoleic acid and gamma linolenic acid. The in-vivo conversion of oleic acid to linoleic acid is carried out by Brassica juncae delta-12 desaturase. The subsequent desaturation of linoleic acid to gamma linolenic acid is catalyzed by the cloned SC-1 delta-6 desaturase. This experiment demonstrates the functional expression of SC-1 delta-6 desaturase in yeast.

1-15. (canceled)

16. An isolated nucleic acid sequence, or fragment thereof, comprising, or complementary to, a nucleotide sequence encoding a polypeptide having Delta-6-desaturase activity, wherein the amino acid sequence of said polypeptide has at least 70%0 identity to an amino acid sequence of SEQ ID NO: 2.

17. The isolated nucleic acid sequence or fragment thereof, according to claim 16, which comprises, or is complementary to, a nucleotide sequence having at least 70% identity to a nucleotide sequence of SEQ ID NO: 1.

18. The isolated nucleic acid sequence or fragment thereof, according to claim 16, comprising, or complementary to, a nucleotide sequence encoding a polypeptide having Delta-6-desaturase activity, wherein the nucleic acid sequence is isolated from Schizochytrium SC1.

19. The isolated nucleic acid sequence of claim 16 wherein said sequence encodes a functionally active Delta-6-desaturase, which utilizes polyunsaturated fatty acid as a substrate.

20. An expression vector comprising the isolated nucleic acid sequence of claim 16 operably linked to a promoter and a termination signal capable of effecting expression of the gene product of said isolated nucleic acid.

21. The expression vector of claim 20, wherein the promoter is a Gall promoter.

22. An expression vector of claim 20, as represented in FIG. 4.

23. A yeast cell transformed with an expression vector of claim 20.

24. A yeast cell transformed with one or more isolated nucleic acid sequences that encode a protein having an activity of desaturating lipid-bound fatty acids, wherein delta-6-desaturases encoded by the nucleic acid sequences convert polyunsaturated fatty acids specifically convert −3 fatty acids.

25. A method of producing polyunsaturated fatty acids comprising the steps of:
(i) screening a cDNA library with a partial delta-4 desaturase gene leading to the identification of a partial cDNA clone,
(ii) screening the BAC library of Schizochytrium SC-I with partial cDNA clone for identification of a positive BAC clone,
(iii) identification and sequencing of the positive BAC clone and further identification of the delta-6 desaturase ORP within the full-length sequence,
(iv) constructing a vector comprising the said isolated nucleic acid sequence operably linked to a regulatory sequence; and
(v) transforming a host yeast cell with said construct, for a time and under conditions sufficient for the expression of the desaturase.

26. A composition comprising at least one polyunsaturated fatty acid produced by a method comprising the steps of:
(i) screening a cDNA library with a partial delta-4 desaturase gene leading to the identification of a partial cDNA clone,
(ii) screening the BAC library of Schizochytrium SC-I with partial cDNA clone for identification of a positive BAC clone,
(iii) identification and sequencing of the positive BAC clone and further identification of the delta-6 desaturase ORP within the full-length sequence,
(iv) constructing a vector comprising the said isolated nucleic acid sequence operably linked to a regulatory sequence; and
(v) transforming a host yeast cell with said construct, for a time and under conditions sufficient for the expression of the desaturase.

27. The composition of claim 26, wherein the said composition is selected from the group consisting of an infant formula, a dietary supplement and a dietary substitute.

28. The composition of claim 26, wherein said composition is formulated to be administered to a human or an animal.

29. The composition of claim 26, wherein said composition is formulated to be administered enterally or parenterally.

30. A method of preventing or treating a condition caused by insufficient intake of polyunsaturated fatty acids comprising administering to a patient a composition of claim 26 in an amount sufficient to effect said prevention or treatment.
